(19) United States
(12) Patent Application Publication
(10) Pub. No.: US 2002/0155518 A1
(43) Pub. Date: Oct. 24, 2002

(54) Method of detecting compounds that control fungal diseases via effects on sporulation

(76) Inventors: Paul J. Kuhn, Yardley, PA (US); Margaret H. Lai, E. Brunswick, NJ (US); Ann B. Orth, Langhorne, PA (US); Helen Azar, Lawrenceville, NJ (US)

Correspondence Address: MERCHANT & GOULD PC, P.O. BOX 2903, MINNEAPOLIS, MN, 55402-0903, US

(21) Appl. No.: 09/905,655

(22) Filed: Jul. 13, 2001

Related U.S. Application Data
(60) Provisional application No. 60/218,194, filed on Jul. 14, 2000.

(51) Int. Cl. C12Q 1/18

(52) U.S. Cl. 435/32

(57) ABSTRACT

The invention relates to a method for identifying chemical or biochemical agents capable of suppressing asexual reproduction in fungi. The method comprises identifying compounds that form a differential zone of development in the area surrounding the test compound on fungal agar plate cultures as compared to normal growth in the surrounding region. Active anti-sporulation compounds are identified based upon the observed suppression of spore formation in the absence of fungal mycelium vegetative growth inhibition. The invention also provides agricultural fungicide and antimycotic compositions, and methods of applying such compositions to control fungal diseases and /or conditions.

### CROSS REFERENCE TO RELATED APPLICATION

This application claims priority to U.S. Provisional Application Ser. No. 60/218,194 filed Jul. 14, 2000 which is herein incorporated by reference in its entirety.

### FIELD OF THE INVENTION

The invention relates to a method for identifying chemical or biochemical agents capable of suppressing asexual reproduction in fungi. The invention also provides agricultural fungicide and antimycotic compositions, and methods of applying such compositions to control fungal diseases and /or conditions.

### BACKGROUND OF THE INVENTION

The development of diseases caused by fungal pathogens can be conveniently separated into a number of phases, which collectively constitute the disease cycle. The scheme shown in FIG. 1 illustrates the four primary phases of fungal disease progression. Interference with one or more of these processes, thereby preventing completion of the cycle, has been the basis for controlling most fungal diseases in agriculture and human medicine.

Agricultural fungicides and antimycotics, identified to date, exert their primary effects by inhibiting the germination, penetration, and/or establishment of infection steps. Inhibition of sporulation represents a central route to effectively control fungal disease since the spores so formed are involved in disease spread to new infection sites and in the development of epidemics. For example, in the case of powdery mildews, which are an important class of fungal plant pathogens, the spores are wind dispersed from the sporulating sites on infected leaf surfaces to healthy plants. The uninfected plants may be adjacent to or physically remote from the plants bearing fungal spores. Therefore, direct inhibition of sporulation may effectively inhibit the spread of such fungal diseases.

In addition to suppressing the production of propagules required for disease spread and the development of epidemics, compounds with an antisporulant mode of action would be expected to have other advantages. For example, they would be likely to have better selectivity than other fungicides since the regulatory and enzymatic pathways involved in sporulation may not be found in plants or mammals. Furthermore, where the mechanism of antisporulant action is mediated via a regulatory element, the chance of developing resistance to the fungicide may be reduced. By contrast, equivalent target enzymes or proteins exist in the host, plant or mammal for many of the fungicides and antimycotics that inhibit earlier phases in fungal disease development. In such cases, selectivity to fungal agents may be more problematic, and thus result in a significantly increased risk of toxicity to the host. Furthermore, resistance to these compounds has often developed rapidly after their introduction, reducing their effectiveness in disease control. Finally, since the processes involved in sporulation have common features in different fungi, compounds with an antisporulant action may provide broad spectrum fungal disease control. This would represent a valuable feature since individual species of plants are typically prone to infection by a variety of different fungal species.

The processes involved in conidiation (synonymous with asexual sporulation or reproduction) in fungi have been most extensively studied in the Ascomycetes Neurospora crassa and Aspergillus nidulans. In the latter fungus, using a combination of classical genetic techniques and molecular approaches, a model has been proposed to describe the complex interrelationships involved in the genetic regulation of development of conidiophores upon which the spores or conidia are born (FIG. 2). Conidiophore development in A. nidulans is initiated within 24 hours after spore germination, making it an ideal test fungus to screen for compounds affecting sporulation. Furthermore, the availability of mutants of, A. nidulans blocked at known steps in the sporulation scheme (Timberlake & Marshall, 1988) may permit follow-up studies to determine more precisely the point at which development is inhibited for each compound identified.

Therefore, there is a need in the art to identify novel fungicide compounds. Specifically, there is a need in the art to identify novel fungicide compounds that inhibit fungal conidiation. More specifically, there is a need in the art to identify novel fungicide compounds that inhibit fungal sporulation. Moreover, there is a need in the art to identify novel fungicide compounds that inhibit fungal sporulation in the absence of affecting fungal vegetative growth. Described herein is a novel method designed to detect compounds that inhibit sporulation by inhibiting, either directly or indirectly, any of the genes and/or proteins involved in the conidiation pathway.

### BRIEF SUMMARY OF THE INVENTION

This invention provides a method for identifying compounds that suppress fungal conidiation that includes the following steps: a) culturing a fungal agent on a solid support containing suitable growth medium,b) adding a test compound to the medium from step “a”, and c) identifying compounds that suppress fungal conidiation by assessing whether the compound from step “b” inhibits the formation of conidia.

The invention also provides a method of determining the mode of action of a compound that suppresses fungal conidiation wherein the method includes: a) culturing a fungal agent on a solid support comprising growth medium, b) adding a test compound to the medium from step “a”, c) identifying compounds that suppress fungal conidiation by assessing whether the compound from step “b” inhibits the formation of conidia, d) culturing a mutant fungal agent on a solid support, e) adding the compound identified in step “c” to the cultured mutant fungal agent from step “d”, and f) determining which mutant fungal agents are sensitive or resistant to the compound from step “c”.

Moreover, the present invention includes a method of using a diagnostic kit for analyzing compounds for suppression of fungal conidiation wherein the method includes: a) providing a test kit comprising a solid support comprising growth medium, and a fungal agent, b) culturing the fungal agent on the solid support, c) adding a test compound to the medium of from step “a”, and d) identifying compounds that supress fungal conidiation by assessing whether the compound from step “c” inhibits the formation of conidia.

Also encompassed by the present invention is a process for production of fungicidal agents comprising the following steps: a) identification of the fungicidal agent by the method of the present invention and b) formulation of the fungicidal agent from step a).

The present invention further provides fungicidal methods and compositions comprising at least one compound identified by the method described herein.

Advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

### BRIEF DESCRIPTION OF THE FIGURES

FIG. 1. Generalized Disease Cycle For Fungal Diseases. The spore, which is the inoculum for disease, comes in contact with the host and germinates by producing a germ tube. Penetration of the host may take place directly, or through wounds, or natural openings. Infection is achieved when the fungus establishes contact with susceptible host cells. The cycle is completed when the fungus reproduces by spores (sporulation).

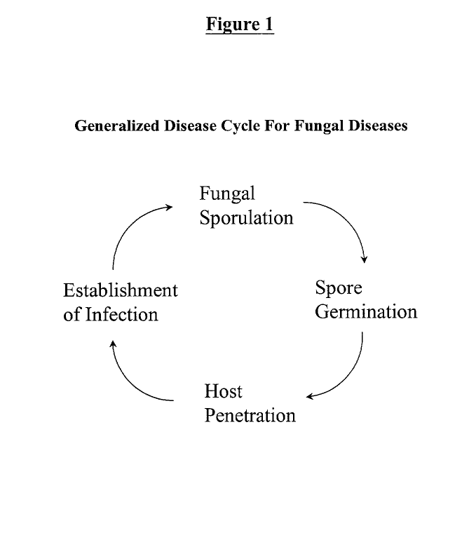

FIG. 2.Genetic Interactions Controlling Asexual Sporulation In A. nidulans. The gene product FluG (fluffy G) is proposed to act as an extracellular signal to activate the sporulation pathway. Genes indicated are flbA,flbB,flbC,flbD,flbE (fluffy A, B, C, D, and E, respectively), brlA (bristleA), abaA (abacusA), and wetA (wet-white). Other genes that regulate conidiation, but which are not shown in the above diagram include stuA (stunted), and medA (medusa). (Scheme based on Adams et al, 1998 and Adams & Yu, 1998).

FIG. 3. Method Used To Detect Antisporulant Compounds In A. nidulans. Test compound is applied on a filter paper disc to the surface of agar medium seeded with spores of

A. nidulans. After incubation for two days, active compounds are identified by the presence of a discolored zone around the filter disc. Conidia production in this zone is greatly reduced compared with the area further out from the filter disc.

FIG. 4. Microscope Slide Assay Used To Characterize Morphological Effects Of Antisporulant Compounds. One half of a slide is coated with a thin layer of unamended PDA, and the other half with agar containing a representative compound with antisporulant activity. The system is inoculated centrally with the fungus, and observations made by light microscopy after incubation for 2-3 days. With antisporulant compounds, differential effects were observed in the formation of conidia and conidia-bearing structures by mycelium depending on the presence or absence of test compound in the area being observed.

### DETAILED DESCRIPTION OF THE INVENTION

The present invention may be understood more readily by reference to the following detailed description of exemplary embodiments of the invention and the examples included therein.

Before the present compounds, compositions, and methods are disclosed and described, it is to be understood that this invention is not limited to specific synthetic methods of making the compounds or compositions described herein that may of course vary. It is also to be understood that the terminology used herein is for the purpose of describing particular embodiments only and is not intended to be limiting.

In this specification and in the claims that follow, reference will be made to a number of terms that shall be defined to have the following meanings: The terms “fungicide” and “fungicidal” are used herein to denote the inhibitive control or modification of undesired fungal growth. Inhibitive control and modification include all deviations from natural development, such as, total killing, growth retardation, desiccation, regulation, stunting, and stimulation.

The term “fungicidally effective amount” is used to denote any amount which achieves such control or modification when applied to the undesired fungus or to the area in which the fungus is growing. “Locus” and “fungal locus” mean a plant, seed, soil, area, material or environment in which fungus is growing or may grow. Exemplary plants and seeds include crop plants and their seeds or nuts, such as vines, wheat, barley, apples, tomatoes, rye, soybeans, oats, rice, maize, lawn, bananas, cotton, coffee, sigar cane, grapevines, fruit species, ornamentals, cucumbers, beans, tomatoes, potatoes and curcubits.

The term “plants” is intended to include seeds, seedlings, germinated seeds, emerging seedlings, plant tissue (e.g., meristematic tissue, root tissue, stem tissue, flower tissue, cotyledon tissue, shoot tissue, callus, etc,), plant cultures, plant cells, established vegetation, including both roots and above-ground portion. “Supressing fungal conidiation” as used herein means that fungal conidiation, sporulation and asexual reproduction are inhibited from natural development. The amount of inhibition from natural development may vary from slight inhibition to total inhibition.

As used throughout, the phrase “treating fungus” is used to mean that fungus and/or a fungal locus has contact with the present compound(s) or composition(s) by application methods known in the art. As such, the compounds and salts of the present invention can be applied in a number of ways, for example, they can be applied, formulated or unformulated, directly to the fungus and/or locus or they can be sprayed on, broadcast, dusted on or applied as a cream or paste formulation or they can be applied as slow release granules (ie by injecting, shanking, chiseling or working into the soil).

The term “agriculturally acceptable salt” is easily determined by one of ordinary skill in the art and includes hydrohalide, acetic, sulfonic, phosphonic, inorganic and organic acid salts as well assalts that can form with, for example, amines, alkali metal bases and alkaline earth metal bases or quaternary ammonium bases, including zwitterions. Suitable alkali metal and alkaline earth metal hydroxides as salt formers include the hydroxides of lithium, sodium, potassium, magnesium or calcium. Illustrative examples of amines suitable for forming ammonium cations are ammonia as well as primary, secondary and tertiary amines such as methylamine, ethylamine, n-propylamine, isopropylamine, the four isomeric butylamines, n-amylamine, isoamylamine, hexylamine, heptylamine, octylamine, nonylamine, decylamine, pentadecylamine, hexadecylamine, heptadecylamine, octadecylamine, methyl ethylamine, methyl isopropylamine, methyl hexylamine, methyl nonylamine, methyl pentadecylamine, methyl octadecylamine, ethyl butylamine, ethyl heptylamine, ethyl octylamine, hexyl heptylamine, hexyl octylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, 10 trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines such as pyridine, quinoline, isoquinoline, morpholine, piperidine, pyrrolidine, indoline, quinuclidine and azepine; primary arylamines such as anilines, methoxyanilines, ethoxyanilines, o-, m- and p-toluidines, phenylenediamines, benzidines, naphthylamines and o-,m- and p-chloroanilines.

The “compounds” referenced herein refer to compounds tested and/or identified using the method of the present invention. Where applicable, the chemical structure of each compound is shown (see Table 3 and elsewhere herein). In addition, the following table may be referenced for each compounds chemical name. Where applicable, each compounds American Chemical Society Registration Number (CAS Reg. No.) is shown.

| Compound | | |
|---|---|---|
| Number | Chemical Name | CAS Reg. No. |
| A | 4-(1,1,3,3-tetramethylbutyl)benzoic | 20457-62-9 |
| | acid | |
| B | (2,3-dibutoxy-4-methoxy-6- | 183725-83-9 |
| | methylphenyl)(2,6- | |
| | dichlorophenyl)methanone | |
| C | (3-bromo-6-methoxy-2- | N/A |
| | methylphenyl)[3,4-dimethoxy-6- | |
| | methyl-2-[(2- | |
| | fluorophenyl)methoxy]phenyl]methanone | |
| D | (2,6-dichlorophenyl)[2-[(4- | 183726-57-0 |
| | fluorophenyl)methoxy]-3,4-dimethoxy- | |
| | 6-methylphenyl]methanone | |
| E | [3,4-dimethoxy-6-methyl-2-[(3- | 221051-42-9 |
| | methylphenyl)methoxy]phenyl](2,6- | |
| | dimethylphenyl)methanone | |
| F | (2,3,4-trimethoxy-6-methylphenyl)(2- | N/A |
| | methyl-1-naphthalenyl)methanone | |
| G | (2-methoxy-6-methylphenyl)(2,3,4- | 220898-62-4 |
| | trimethoxy-6-methylphenyl)methanone | |
| H | [3,4-dimethoxy-6-methyl-2-[(2,4,6- | 221051-21-4 |
| | trifluorophenyl)methoxy]phenyl](2,6- | |
| | dimethylphenyl)methanone | |
| I | (2-chloro-6-methoxyphenyl)[3,4- | 220902-60-3 |
| | dimethoxy-6-methyl-2-[(3- | |
| | methylphenyl)methoxy]phenyl]methanone | |
| J | (3-bromo-2-chloro-6- | 220898-85-1 |
| | methoxyphenyl)(2,3,4-trimethoxy-6- | |
| | methylphenyl)methanone | |
| K | [4-(cyclopentyloxy)-5-methoxy-2- | N/A |
| | methylphenyl](2,6- | |
| | dichlorophenyl)methanone | |
| L | (2,6-dimethylphenyl)(2,3,4-trimethoxy- | N/A |
| | 6-methylphenyl)methanone | |
| M | (3-bromo-6-methoxy-2- | 220899-03-6 |
| | methylphenyl)(2,3,4-trimethoxy-6- | |
| | methylphenyl)methanone | |
| N | (3-chloro-6-hydroxy-2- | 252955-12-7 |
| | methylphenyl)(2,3,4-trimethoxy-6- | |
| | methylphenyl)methanone | |
| O | [3,4-dimethoxy-6-methyl-2- | 221051-20-3 |
| | (phenylmethoxy)phenyl](2,6- | |
| | dimethylphenyl)methanone | |
| P | (2-chloro-6-methoxy-3- | N/A |
| | methylphenyl)[2,4-dimethoxy-6- | |
| | methyl-3-(pentyloxy)phenyl]methanone | |
| Q | (3-chloro-2,6-dimethylphenyl)(2,3,4- | 221051-17-8 |
| | trimethoxy-6-methylphenyl)methanone | |
| R | (3-chloro-6-methoxy-2- | 220900-12-9 |
| | methylphenyl)(2,3,4-trimethoxy-6- | |
| | methylphenyl)methanone | |
| S | (3-bromo-6-methoxy-2- | N/A |
| | methylphenyl)[3,4-dimethoxy-6- | |
| | methyl-2-[(3- | |
| | methylphenyl)methoxy]phenyl]methanone | |
| T | [6-methoxy-2-methyl-3- | 220900-85-6 |
| | (trifluoromethyl)phenyl](2,3,4- | |
| | trimethoxy-6-methylphenyl)methanone | |
| U | (2,6-dichlorophenyl)[5-methoxy-2- | N/A |
| | methyl-4-(1- | |
| | methylbutoxy)phenyl]methanone | |
| V | (2,6-dichlorophenyl)[2-[(2- | 183726-56-9 |
| | fluorophenyl)methoxy]-3,4- | |
| | dimethoxy-6-methylphenyl]methanone | |
| W | (2,6-dichlorophenyl)[3,4-dimethoxy-6- | N/A |
| | methyl-2-(1- | |
| | oxopropoxy)phenyl]methanone or | |
| | propanoic acid, 2-(2,6- | |
| | dichlorobenzoyl)-5,6-dimethoxy-3- | |
| | methylphenyl ester | |
| X | [3,4-dimethoxy-6-methyl-2-[(2- | N/A |
| | fluorophenyl)methoxy]phenyl](2- | |
| | methyl-1-naphthalenyl)methanone | |
| Y | (3-bromo-6-methoxy-2- | N/A |
| | methylphenyl)[4-methoxy-6-methyl-2- | |
| | (phenylmethoxy)-3- | |
| | propoxyphenyl]methanone | |
| Z | (3-bromo-6-methoxy-2- | N/A |
| | methylphenyl)[4-methoxy-6-methyl-2- | |
| | (1-oxopropoxy)-3- | |
| | propoxyphenyl]methanone or | |
| | Propanoic acid, 2-(3-bromo-6-methoxy- | |
| | 2-methylbenzoyl)-5-methoxy-3-methyl- | |
| | 6-propoxyphenyl ester | |
| AA | (3-bromo-6-methoxy-2- | N/A |
| | methyiphenyl)[3,4-dimethoxy-6- | |
| | methyl-2-[(2- | |
| | methylphenyl)methoxy]phenyl]methanone | |
| BB | (3-bromo-6-methoxy-2- | N/A |
| | methylphenyl)[4-methoxy-6-methyl-3- | |
| | propoxy-2-[[4-(1,1- | |
| | dimethylethyl)phenyl] | |
| | methoxy]phenyl]methanone | |
| CC | (3-bromo-6-methoxy-2- | N/A |
| | methylphenyl)[4-methoxy-6-methyl-3- | |
| | propoxy-2-[[4- | |
| | (trifluoromethyl)phenyl]methoxy]phenyl] | |
| | methanone | |
| DD | (3-chloro-6-methoxy-2- | N/A |
| | methylphenyl)(2-hydroxy-4-methoxy- | |
| | 6-methyl-3-propoxyphenyl)methanone | |
| EE | (3-bromo-6-methoxy-2- | N/A |
| | methylphenyl)[3,4-dimethoxy-6- | |
| | methyl-2-[(4- | |
| | chlorophenyl)methoxy]phenyl]methanone | |
| FF | (3-chloro-6-methoxy-2- | 220900-68-5 |
| | methylphenyl)(2,4-dimethoxy-6- | |
| | methyl-3-propoxyphenyl)methanone | |

This invention relates to a novel screening method for identifying compounds that specifically inhibit the formation of conidia in fungi. The screen will detect compounds that act at any of the points in the conidiation pathway and enzymes or other gene products that mediate regulation of conidia formation.

Therefore, one of the main objectives of the invention concerned the development of a method for identifying inhibitory compounds of fungal asexual spore formation that could potentially act as agricultural fungicides or antimycotics.

In the developed method, test samples are applied to filter paper discs that are then placed on a solidified agar medium seeded with a spore inoculum of a suitable filamentous fungus, such as Aspergillus nidulans.

Suitable filamentous fungi of the method of the present invention may be a member selected from the group consisting of: Aspergillus, Penicillium, Botrytis, and Alternaria. Preferably, the fungal agent of the present invention is Aspergillus nidulans. Other fungal species may be substituted for the fungal agents of the present invention, particularly other species of Aspergillus like Aspergillus niger, Aspergillus fumigatus, Aspergillus flavus, Aspergillus oryzae. Additional suitable species are known in the art and are described elsewhere herein.

Alternatively, test samples may be applied directly to the agar surface and/or mixed within the agar medium. After incubation of cultures, active compounds are identified by the production of a differential zone around either the filter disc or the area of agar to which the compound had been applied. Such an observed zone is due to suppression of conidiation. Spores are colored entities, and their specific inhibition allows for an easily discernable color change in the fungus's appearance. An essential tenet of the method of the present invention concerns the identification of compounds that inhibit fungal conidiation and/or sporulation in the absence of inhibiting fungal vegetative mycelial growth. However, slight to moderate vegetative mycelial growth inhibition may be observed and expected in certain circumstances. Moreover, the method has also been shown to work with other species of Aspergillus. Therefore, the invention encompasses the substitution of Aspergillus nidulans with other species of Aspergillus, which include, but are not limited to Aspergillus niger, Aspergillus fumigatus, Aspergillus flavus, Aspergillus oryzae in addition to other fungal organisms described herein.

The method of the present invention for identifying compounds that suppress conidiation comprises the following steps: a) culturing a fungal agent on a solid support comprising suitable growth medium, b) adding a test compound to the medium of the cultured fungal agent from step “a”, and c) identifying compounds that suppress fungal conidiation by assessing whether the compound from step “b” inhibits the formation of conidia.

The compounds may be assessed in step “c” by observing a discolored zone on the medium in an area proximate to the test compound. Furthermore, the method can comprise an additional step “d” which involves determination of reduced spore. The determination of reduced spore production can be performed visually, e.g. by viewing the discolored zone under a loop or microscope, wherein the use of microscope is preferred.

The above mentioned assay can be completed by the use of control compounds. A control compound is herein defined as a compound which inhibits sporulation according to the assay of the present invention. The control compound is applied in the same way as the test compound. By comparing the discolored zones after step c) the efficiency of test compounds can be checked.

In a preferred embodiment the assay is performed in a high-throughput screen.

The solid support of the method of the present invention may be a member selected from the group consisting of: a Petri dish, a square bioassay plate, and a glass microscope slide containing suitable growth medium. Additional solid supports are known in the art that could be readily substituted for the aforementioned supports by the skilled artisan. Such substitutions are encompassed by the present invention.

The invention also encompasses inhibitors of fungal conidiation identified according to the method of the present invention. Such inhibitors may inhibit conidiation and/or sporulation. Preferably, the inhibitors of the present invention inhibit conidiation and/or sporulation in the absence of inhibiting vegetative growth.

The method of the present invention encompasses the further step of determining which test compounds have fungicidal effects. Specifically, the method of the present invention encompasses the further step of determining which inhibitors of the present invention have fungicidal activity. As mentioned elsewhere herein, the compounds identified as inhibitors of fungal conidiation and/or sporulation by the method of the present invention, may have fungicidal activity. As a result, it is expected that compounds identified by the method of the present invention are capable of serving as fungicides, either directly or indirectly.

Once a compound is identified as a fungal conidiation and/or sporulation inhibitor, a variety of techniques can be applied to determine whether the compound has fungicidal activity for one or more fungal species and/or host plants. For example, the fungal organisms tested may be any fungus known in the art, though preferably the fungal organisms described elsewhere herein. Moreover, the host plants tested may be any plant known in the art, though preferably, the plants referenced elsewhere herein. In one example, the experimental compound of the present invention is solubilized in a liquid formulation to the required test concentration (ppm as wt a.i.:wt soil). The formulation may or may not contain a surfactant. The resulting test solution containing the formulated (if required) compound of the present invention, is applied exogenously to the plant, and/or soil, and allowed to dry. Later the same day, the treated plant foliage, and/or roots, are inoculated with an appropriate plant fungal pathogen. The plant fungal pathogen may be one, or more, fungal organisms known in the art. The inoculated plants are kept under conditions conducive to infection for 1-5 days, and then are subjected to conditions conducive to post-infection disease development for an additional 1 to 35 days depending upon the unique requirements of the fungal pathogen. Once disease signs/symptoms are observed, the compounds of the present invention are evaluated for an observable reduction in disease level relative to an untreated, inoculated control. The skilled artisan would appreciate that the specific innoculation conditions may vary from one organism to another and may need to be empirically determined. The invention encompasses the application of one or more compounds of the present invention to the liquid formulation and/or soil above.

The precise composition of the liquid formulation may be empirically determined based upon the unique stability requirements of the compounds of the present invention. For example, the compound of the present invention may require addition of known stabilizing agents, dispersing agents, surfactants, etc.

Once a compound of the present invention is determined to confer control to fungal infestation, the positive compound of the present invention can then be subjected to secondary evaluations to identify the effective concentration. For example, the test compounds of the secondary evaluations could be prepared with a liquid formulation comprising one or more compounds of the present invention, at a rate of application (ppm as wt a.i.:wt soil) equal to 1×, ⅓×, and/or 1/30 the rate originally tested in the primary screen, for example.

The skilled artisan would appreciate that the above example is exemplary, and should not be construed as limiting. Moreover, the skilled artisan would appreciate that other methods are known in the art that may be more applicable, or preferred, in certain circumstances. Such methods could readily be substituted for the methods outlined above.

The invention encompasses fungicidal compositions comprising as fungicidal agent an inhibitor identified according to the method of the present invention determined to have fungicidal activity like compound “A”. The invention also encompasses fungicidal compositions comprising more than one inhibitor identified by the method of the present invention.

Thus, the present invention encompasses a method of use for production of fungicidal agents comprising the following steps: a)identification of the fungicidal agent by the method of the present invention as described above; b) preparing a fungicidal composition comprising the fungicidal agent identified in step a).

The present invention further encompasses fungicidal compostions prepared by the method mentioned above.

The preparation of fungicidal compositions, the formulation of the fungicidal agent, is described herein elsewhere.

Fungicidal compositions of the present invention may further comprise stabilizers, surfactants, diluants, protectants, in addition to, one or more additional fungicidal compounds.

The present invention encompasses a method of controlling undesired fungal growth by applying to a fungi or a locus where control is desired a fungicidal composition of the present invention.

Another aspect of the present invention is the use of mutants defective at various sites in the conidiation pathway, to identify the point in the pathway at which the antisporulant effect of an active compound originates. Specifically, the present invention relates to a method of determining the mode of action of the fungal conidiation inhibitor identified according to the method of the present invention. Such a method may comprise the following steps: a)identifing compounds that suppress fungal conidiation according to the method described above; b) culturing a mutant fungal agent on a solid support; c) adding a compound identified in step “a” to the cultured mutant fungal agent from step “b”, and d) determining which mutant fungal agents are sensitive or resistant to the compound from step “a”.

The mutant fungal agents for the method of determining the mode of action of the fungal conidiation inhibitor are preferably mutant fungal agents of the fungal conidiation pathway. Specifically, the mutant fungal agents are a member of the mutants selected from the group consisting of: fluG, flbA, flbE, flbD, flbB, flbC, brIA, abaA, wetA, and/or other fungal mutants as identified. More preferred are mutant fungal agents belonging to the fungal species selected from the group consisting of: Aspergillus, Penicillium, Botrytis, and Alternaria. Additional mutant fungal agents are known in the art and are encompassed by the present invention.

According to still another aspect, the invention describes the utility of compounds active as antisporulants in the control of powdery mildews, an important class of fungal pathogens of crops.

The invention also encompasses inhibitors of fungal conidiation identified according to the method of the present invention and methods of producing fungicidal agents by identifying such inhibitors and formulation into a fungicidal agent. Fungal conidiation inhibitors may inhibit conidiation and/or sporulation. Preferably, the inhibitors of the present invention inhibit conidiation and/or sporulation in the absence of inhibiting vegetative growth. The method of the present invention encompasses the further step of determining which test compounds have fungicidal effects. Specifically, the method of the present invention encompasses the further step of determining which inhibitors of the present invention have fungicidal activity.

Also included in the present invention are compounds having the formula (I):

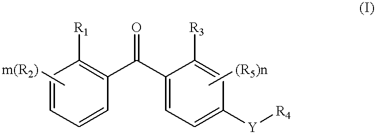

wherein:

R₁ is hydrogen, halogen, hydroxy, C₁-C₆ alkyl, C₁-C₆ alkoxy, C₁-C₆ haloalkyl, or benzyl fused at the R₁ position;

R₂ is hydrogen, halogen, hydroxy, C₁-C₆ alkyl, C₁-C₆ alkoxy, or C₁-C₆ haloalkyl;

R₃ is hydrogen, halogen, hydroxy, carbonyl, C₁-C₆ alkyl, C₁-C₆ alkoxy, or C₁-C₆ haloalkyl, wherein the alkyl, alkoxy, or haloalkyl group is unsubstituted or substituted with benzyl or phenyl and the benzyl or phenyl is unsubstituted or substituted with one or more of the following: halogen, hydroxy, C₁-C₆ alkyl, C₁-C₆ alkoxy, or C₁-C₆ haloalkyl at any position on the aromatic structure;

R₄ is hydrogen, C₁-C₆ alkyl, C₁-C₆ alkoxy, or C₁-C₆ haloalkyl;

R₅ is halogen, hydroxy, C₁-C₆ alkyl, C₁-C₆ alkoxy, or C₁-C₆ haloalkyl;

Y is O, N or S;

m is 0, 1,2,or3;and

n is 0, 1, 2, or 3;

and agriculturally acceptable salts and esters thereof.

Desirably, the formula (I) compound has the following substituents R₁ is halogen, C₁-C₃ alkyl, C₁-C₃ alkoxy, or benzyl fused at the R₁ position;

R₂ is halogen, hydroxy, C₁-C₃ alkyl, C₁-C₃ alkoxy, or C₁-C₃ haloalkyl;

R₃ is hydroxy, carbonyl, C₁-C₄ alkyl, or C₁-C₄ alkoxy, wherein the alkyl or alkoxy, group is unsubstituted or substituted with benzyl or phenyl and the benzyl or phenyl is unsubstituted or substituted with one or more of the following: halogen, C₁-C₄ alkyl, or C₁-C₄ haloalkyl at any position on the aromatic structure;

R₄ is C₁-C₆ alkyl or C₁-C₆ alkoxy;

R₅ is C₁-C₆ alkyl or C₁-C₆ alkoxy;

Y is O;

m is 1 or 2;and

n is 1 or 2;

and agriculturally acceptable salts and esters thereof.

More desirable compounds include the following:

(2,3-dibutoxy-4-methoxy-6-methylphenyl)(2,6-dichlorophenyl)methanone;

(3-bromo-6-methoxy-2-methylphenyl) [3 ,4-dimethoxy-6-methyl-2-[(2-fluorophenyl)methoxy]phenyl]methanone;

(2,6-dichlorophenyl) [2-[(4-fluorophenyl)methoxy]-3 ,4-dimethoxy-6-methylphenyl]methanone;

[3 ,4-dimethoxy-6-methyl-2-[(3 -methylphenyl)methoxy]phenyl](2,6-dimethylphenyl)methanone;

(2,3 ,4-trimethoxy-6-methylphenyl)(2-methyl- 1 -naphthalenyl)methanone;

(2-methoxy-6-methylphenyl)(2,3,4-trimethoxy-6-methylphenyl)methanone;

[3 ,4-dimethoxy-6-methyl-2-[(2,4,6-trifluorophenyl)methoxy]phenyl](2,6-dimethylphenyl)methanone;

(2-chloro-6-methoxyphenyl) [3 ,4-dimethoxy-6-methyl-2-[(3 -methylphenyl)methoxy]phenyl]methanon

(3-bromo-2-chloro-6-methoxyphenyl)(2,3,4-trimethoxy-6-methylphenyl)methanone;

[4-(cyclopentyloxy)-5-methoxy-2-methylphenyl](2,6-dichlorophenyl)methanone;

(2,6-dimethylphenyl)(2,3 ,4-trimethoxy-6-methylphenyl)methanone;

(3-bromo-6-methoxy-2-methylphenyl)(2,3 ,4-trimethoxy-6-methylphenyl)methanone;

(3 -chloro-6-hydroxy-2-methylphenyl)(2,3 ,4-trimethoxy-6-methylphenyl)methanone;

[3 ,4-dimethoxy-6-methyl-2-(phenylmethoxy)phenyl](2,6-dimethylphenyl)methanone;

(2-chloro-6-methoxy-3 -methylphenyl) [2,4-dimethoxy-6-methyl-3-(pentyloxy)phenyl]methanone;

(3-chloro-2,6-dimethylphenyl)(2,3,4-trimethoxy-6-methylphenyl)methanone;

(3-chloro-6-methoxy-2-methylphenyl)(2,3,4-trimethoxy-6-methylphenyl)methanone;

(3-bromo-6-methoxy-2-methylphenyl) [3 ,4-dimethoxy-6-methyl-2-[(3 -methylphenyl) methoxy]phenyl]methanone;

[6-methoxy-2-methyl-3 -(trifluoromethyl)phenyl](2, 3 ,4-trimethoxy-6-methylphenyl)methanone;

(2,6-dichlorophenyl) [5-methoxy-2-methyl-4-( 1-methylbutoxy)phenyl]methanone;

(2,6-dichlorophenyl)[2-[(2-fluorophenyl)methoxy]-3 ,4-dimethoxy-6-methylphenyl]methanone;

(2,6-dichlorophenyl)[3 ,4-dimethoxy-6-methyl-2-( 1-oxopropoxy) phenyl]methanone or propanoic acid, 2-(2,6-dichlorobenzoyl)-5,6-dimethoxy-3-methylphenyl ester;

[3 ,4-dimethoxy-6-methyl-2-[(2-fluorophenyl)methoxy]phenyl](2-methyl- 1-naphthalenyl)methanone;

(3-bromo-6-methoxy-2-methylphenyl)[4-methoxy-6-methyl-2-(phenylmethoxy)-3 -propoxyphenyl]methanone;

(3-bromo-6-methoxy-2-methylphenyl)[4-methoxy-6-methyl-2-(1-oxopropoxy)-3 -propoxyphenyl]methanone or Propanoic acid, 2-(3-bromo-6-methoxy-2-methylbenzoyl)-5 -methoxy-3-methyl-6-propoxyphenyl ester;

(3 -bromo-6-methoxy-2-methylphenyl)[3 ,4-dimethoxy-6-methyl-2-[(2-methylphenyl)methoxy]phenyl]methanone;

(3 -bromo-6-methoxy-2-methylphenyl) [4-methoxy-6-methyl-3 -propoxy-2-[[4-(1,1 dimethylethyl)phenyl]methoxy]phenyl]methanone;

(3-bromo-6-methoxy-2-methylphenyl)[4-methoxy-6-methy1-3 -propoxy-2-[[4-(trifuoromethyl)phenyl]methoxy]phenyl]methone;

(3-chloro-6-methoxy-2-methylphenyl)(2-hydroxy-4-methoxy-6-methyl-3 -propoxyphenyl)methanone;

(3 -bromo-6-methoxy-2-methylphenyl) [3 ,4-dimethoxy-6-methyl-2-[(4-chlorophenyl) methoxy]phenyl]methanone; and

(3-chloro-6-methoxy-2-methylphenyl)(2,4-dimethoxy-6-methyl-3-propoxyphenyl)methanone.

Moreover, the present invention includes compound “A” having the formula (II):

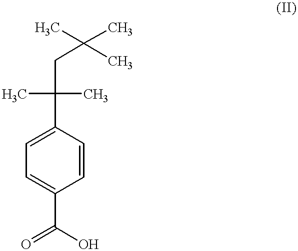

and agriculturally acceptable salts and esters thereof.

The invention encompasses fungicidal compositions comprising a fungicidally effective amount of an inhibitor identified according to the method of the present invention or a compound having the formula (I) or (II). The invention also encompasses fungicidal compositions comprising more than one inhibitor identified by the method of the present invention.

Fungicidal compositions of the present invention may further comprise carriers, stabilizers, surfactants, diluants, and/or protectants, in addition to, one or more additional fungicidal compounds.

The present invention encompasses a method of controlling undesired fungal growth by applying to a locus where control is desired a fungicidal composition of the present invention.

Moreover, the present invention relates to a method of using an inhibitor of the present invention in a diagnostic kit comprising incubating a sample in the presence of said inhibitor and analyzing the results. The sample may be a member selected from the group consisting of: fungal organism, fungal tissue, fungal cells, fungal protein, fungal DNA, fungal RNA, fungal cDNA, fungal genomic DNA, etc. Alternatively, the sample may be a member selected from the group consisting of: plants, plant tissue, plant cells, plant protein, plant DNA, plant RNA, plant cDNA, plant genomic DNA, etc. Alternatively, the sample may comprise any combination of the above.

Another aspect of the present invention is the use of mutants defective at various sites in the conidiation pathway, to identify the point in the pathway at which the antisporulant effect of an active compound originates. Specifically, the present invention relates to a method of determining the mode of action of the fungal conidiation inhibitor identified according to the method of the present invention. Such a method may comprise the following steps: i. culturing a mutant fungal agent on a solid support; ii. adding the fungal conidiation inhibitor identified according to the method of the present invention or a control compound to the center of the cultured mutant fungal agent from step “i.”; iii. determining whether the compound inhibits the formation of conidia by observing the formation of a discolored zone surrounding the point of application of said test compound; and iv. noting which mutant fungal agents are sensitive or resistant to the inhibitor identified according to the method of the present invention.

The mutant fungal agents for the method of determining the mode of action of the fungal conidiation inhibitor are preferably mutant fungal agents of the fungal conidiation pathway. Specifically, the mutant fungal agents are fluG, flbA, fibE, flbD, flbB, flbC, brlA, abaA, wetA, stuA, medA, and/or other fungal mutants as identified. More preferred are mutant fungal agents belonging to the fungal species selected from the group consisting of: Aspergillus, Penicillium, Botrytis, and Alternaria. Additional mutant fungal agents are known in the art and are encompassed by the present invention.

According to still another aspect, the invention describes the utility of compounds active as antisporulants in the control of powdery mildews, an important class of fungal pathogens of crops.

Uses for the Method of the Present Invention

The method of the present invention is useful for identifying compounds capable of inhibiting, either directly or indirectly, fungal sporulation in the absence of affecting fungal vegetative growth.

The method of the present invention is useful for identifying compounds that inhibit, either directly or indirectly, steps in the conidiation pathway. Specifically, the method of the present invention is useful for identifying compounds that inhibit, either directly or indirectly, the following non-limiting genes, or alternatively the proteins encoded by these genes, involved in the conidiation pathway: fluG, flbA, flbE, flbD, flbB, flbc, brIA, abaA, wetA, and/or other steps as identified or known in the art.

The method of the present invention may also be useful for identifying other genes, or alternatively, the proteins encoded by these genes that may modulate, either directly or indirectly, the cellular expression levels or activity of proteins involved in the conidiation pathway.

Uses for the Compounds Identified by the Method of the Present Invention

The compounds identified by the method of the present invention may be useful for inhibiting conidiation of a number of fungal pathogens.

The compounds identified by the method of the present invention are useful for identifying compounds capable of inhibiting, either directly or indirectly, fungal sporulation in the absence of affecting fungal vegetative growth of various fungal agents.

Compounds identified by the method of the present invention are useful for identifying compounds that inhibit, either directly or indirectly, steps in the conidiation pathway. Specifically, compounds identified by the method of the present invention may be useful for identifying compounds that inhibit, either directly or indirectly, the following non-limiting genes, or alternatively the proteins encoded by these genes, involved in the conidiation pathway: fluG, flbA, flbE, flbD, flbB, flbc, brlA, abaA, and/or wetA.

Since conidiation and sporulation represent essential steps in fungal growth and disease progression, compounds capable of inhibiting either of these steps, may be useful as a potent fungicide. Therefore, compounds identified by the method of the present invention may be useful as a fungicide for a number of fungal pathogens. Examples of fungal pathogens for which a compound identified by the present invention may be useful in inhibiting includes, but is not limited to, the following: Actinomycetales (e.g., Corynebacterium, Mycobacterium, Norcardia), Cryptococcus neoformans, Aspergillus, Myxomycota (e.g., Myxomycetes (Fuligo, Muciliago, Physarum, Physarales, etc), and Plasmodiophoromycetes (Plasmodiophora (e.g., P. brassicae), Polymyxa (e.g., P. gramminis, etc.), Spongospora (e.g., S. subteranea, etc. )), Eumycota (e.g., Mastigomycotina, Chrytridiomycetes (e.g., Olpidium brassicae, Physoderma maydia, Synchytrium endobioticum, Urophlyctis alfalfae, etc.), Oomycetes, Saprolegniales (e.g., Aphanomyces, etc.), Peronosporales, Pythiaceae (e.g., Pythium, Phytophthora infestans, etc.), Albuginaceae (e.g.,Albugo candida, etc.), Peronosporaceae (e.g., Plasmopara viticola,Peronospora nicotianae, Bremia lactucae, Sclerospora graminicola, and Pseudoperonospora cubensis etc.), Zygomycotina, Zygomycetes, Mucorales (e.g., Rhizopus, Choanephora cucurbitarum, etc.), Endogonales, Endogone, Ascomycotina, Hemiascomycetes, Endomycetales (e.g., Saccharomyces cerevisiae, etc.), Taphrina, Pytenomycetes, Erysiphales (e.g., Erysiphe, Microsphaera, Podosphaera leucotricha, Spaerotheca pannosa, Uncinula necator etc.), Sphaeriales (e.g., Botryosphaeria obtusa, Ceratocystis, Diaporthe, Endothia parasitica, Eutypa armeniacae, Glomerella cingulata, Gnomonia, Hypoxylon mammatum, Rosellinia, Valsa, Xylaria, etc.), Hypocreales (e.g., Claviceps purpurea, Gibberella, Nectria, etc.), Loculoascomycetes, Myriangiales (e.g., Elsinoe, etc.), Dothideales (e.g., Capnodium, Didymella, Guignardia bidwelii, Microcyclus elei, Plowrightia morbosum, etc.) Pleosporales (e.g., Cochliobolus sativus, Gaeumannomyces graminis, Pyrenophora, Venturia inaequalis, etc.), Discomycetes, Phacidiales (e.g.,Rhytisma acerinum), Helotiales (e.g., Diplocarpos rosae, Higginsia hiemalis, Lophodermium, Monilinia fructicola, Pseudopeziza trifolii, Sclerotinia sclerotiorum, etc.), Deuteromycotina, Coelomycetes, Sphaeropsidales (e.g., Ascochyta pisi, Coniothyrium, Cytospora, Diplodia maydis, Phoma lingam, Phomopsis, Phyllosticta, Septoria apii, etc.), Melanconiales (e.g., Celletotrichum, Coryneium beijerincki, Cylindrosporium, Gloeosporium, Marssonina, Melanconium fuligenum, Sphaceloma, etc.), Hyphomycetes, Hyphales (e.g., Alternaria, Asperigillus, Bipolaris, dreschslerea, Excerophilum, Botrytis cinerea, Cercospora, Fulvia fulva, Fusarium, Geotrichum candidum, Graphium ulmi, Peniciuum, Phymatotrichum omnivorum, Pyricularia, Spilocaea, Theilaviopsis basicola, Trichoderma, Verticillum, etc.), Agonomycetes, Agonomycetales (e.g., Rhizoctonia, Sclerotium, etc.), Basidomycotina, Hemibasidiomycetes, Ustilaginales (e.g., Sphaceltheca, Tilletia, Urocystis cepulae, Ustilago, etc.), Uredinales (e.g., Cronartium, Gymnosporangium Juniperi-virginianae, Melampsora lini, Phragmidium, Puccinia, Uromyces appendiculatus, etc.), Hymenomycetes, Exobasidiales (e.g., Exobasidium, etc.), Aphyllochorales (e.g., Aethalia, Corticium, Heterobasidum, Lenzites, Peniophora, Polyporus, Poria, Schizophyllum, Stereum, etc.), Tulasnellales (e.g., Thanatephorus, Typhula, etc.), and Agaricales (e.g., Armillaria mellea, Marasmius, Pholiota, Pleurotus, etc.). Additional fungal pathogens are known in the art (see, for example, G. N., Agrios, supra, G. C. Ainsworth, in “Fungal Diseases of Animals”, Commonwealth Agricultural Bureaux, Farmham Royal Bucks, England, (1959), and Jones, T. C., in “Veterinary Pathology”, 4^{th}Edition, Lea and Febiger, Philadelphia, (1972)).

Fungal pathogens falling within any of the aforementioned divisions, subdivisions, classes, orders, genus's, or species can cause a variety of plant diseases or symptoms, generally including, but not limited to: necrosis, plant death, cell death, hypotrophy, hypoplasmia, stunting, hyperplasia (e.g., clubroot, galls, warts, witches brooms, leaf curls, etc.), tumors, leaf spots, blight, cankers, dieback, root rot, damping off, basal stem rot, soft rots, dry rots, anthracnose, scab, decline, wilt, rust, mildew, and smut; and specifically, fructifications, powdery scab of potato, clubroot of crucifers, black wort of potato, crown wart of alfalfa, brown spot of corn, seed rot, seedling damping off, root and stem rot, blight, tuber rot, white rust, upper side, downy mildews, oospores on soybean seed, rhizopus soft rots, rhizopus fruit rot, choanephora squash rot, bread mold, bud rot, stem rot, collar rot, crown rot, trunk rot, black pod disease, late blight of potatoes, Anthracnose diseases, Colletotrichum diseases, onion smudge, ergot, Botrytis diseases, vascular wilts, Dutch Elm disease, Gibberella diseases, Sclerotinia diseases, Rhizoctonia diseases, Sclerotium diseases, postharvest decay of fruits and vegetables, decreased photosynthesis, decreased chlorophyll per leaf, decreased chlorophyll efficiency, decrease in plant hormone production, decreased growth rate, decreased soluble nitrogen, decreased carbohydrate levels, either an increase or decrease in respiration, aberant plant metabolism, decrease water translocation, decreased nutrient retention, increased transpiration, reduced yields, modulate transcription of the plant, modulate translation of the plant, and aberrant cellular metabolism. Therefore compounds identified by the method of the present invention, can be used, either directly or indirectly, to prevent, and/or confer resistance to any of these symptoms or diseases.

As referenced elsewhere herein, fungal pathogens are also known to contribute to significant diseases and/or disorders in animals, and particularly in humans. Since sporulation represents a basal requirement for the spread of fungal diseases and/or disorders, the compounds identified by the method of the present invention may be useful in inhibiting fungal diseases and/or disorders in animals, and particularly in humans. Examples of fungal diseases and/or disorders afflicting animals and humans include, but are not limited to, the following: ringworm, Aspergillosis, placental caruncle, mucormycotic ulceration, mucormycosis, mycotic abortion, actinomycosis, nocardiosis, cryptococcosis, epizootic lymphangitis, sporotrichosis, histoplasmosis, blastomycosis, gangrenous ergotism, mucirmycosis, coccidiosis, coccidioidomyosis, haplomycosis, rhinosporidiosis, streptothricosis, moniliasis, strawberry foot rot, toxicoses, ergotism, and/or mycotic dermatitis. Additional fungal diseases and/or disorders afflicting animals are known in the art (see, for example, Ainsworth, G. C., and Austwich, P. K. C., in “Fungal Diseases of Animals”, Commonwealth Agricultural Bureaux, Farnham Royal, Bucks, England, (1959)).

Moreover, the present invention encompasses testing various host plants, and the plant cells, plant tissues, and/or plant seeds derived from said host plants, susceptible to fungal infection, for the ability of the compounds identified by the method of the present invention to serve the role as a fungicide. Non-limiting examples of suitable recipient plants for testing the compounds of the present invention for fungicidal activty are listed in the Table below. Examples of methods that may be used for testing the compounds identified by the present invention are provided, for example, in Example 5 and elsewhere herein.

RECIPIENT PLANTS

| COMMON NAME | FAMILY | LATIN NAME |
|---|---|---|
| Maize | Gramineae | Zea mays |
| Maize, Dent | Gramineae | Zea mays dentiformis |
| Maize, Flint | Gramineae | Zea mays vulgaris |
| Maize, Pop | Gramineae | Zea mays microsperma |
| Maize, Soft | Gramineae | Zea mays amylacea |
| Maize, Sweet | Gramineae | Zea mays amyleasaccharata |
| Maize, Sweet | Gramineae | Zea mays saccharate |
| Maize, Waxy | Gramineae | Zea mays ceratina |
| Wheat, Dinkel | Pooideae | Triticum spelta |
| Wheat, Durum | Pooideae | Triticum durum |
| Wheat, English | Pooideae | Triticum turgidum |
| Wheat, Large Spelt | Pooideae | Triticum spelta |
| Wheat, Polish | Pooideae | Triticum polonium |
| Wheat, Poulard | Pooideae | Triticum turgidum |
| Wheat, Singlegrained | Pooideae | Triticum monococcum |
| Wheat, Small Spelt | Pooideae | Triticum monococcum |
| Wheat, Soft | Pooideae | Triticum aestivum |
| Rice | Gramineae | Oryza sativa |
| Rice, American Wild | Gramineae | Zizania aquatica |
| Rice, Australian | Gramineae | Oryza australiensis |
| Rice, Indian | Gramineae | Zizania aquatica |
| Rice, Red | Gramineae | Oryza glaberrima |
| Rice, Tuscarora | Gramineae | Zizania aquatica |
| Rice, West African | Gramineae | Oryza glaberrima |
| Barley | Pooideae | Hordeum vulgare |
| Barley, Abyssinian | Pooideae | Hordeum irregulare |
| Intermediate, also | | |
| Irregular | | |
| Barley, Ancestral | Pooideae | Hordeum spontaneum |
| Tworow | | |
| Barley. Beardless | Pooideae | Hordeum trifurcatum |
| Barley, Egyptian | Pooideae | Hordeum trifurcatum |
| Barley, fourrowed | Pooideae | Hordeum vulgare polystichon |
| Barley, sixrowed | Pooideae | Hordeum vulgare hexastichon |
| Barley, Tworowed | Pooideae | Hordeum distichon |
| Cotton, Abroma | Dicotyledoneae | Abroma augusta |
| Cotton, American | Malvaceae | Gossypium hirsutum |
| Upland | | |
| Cotton, Asiatic Tree, also | Malvaceae | Gossypium arboreum |
| Indian Tree | | |
| Cotton, Brazilian, also, | Malvaceae | Gossypium barbadense |
| Kidney, and, | | brasiliense |
| Pemambuco | | |
| Cotton, Levant | Malvaceae | Gossypium herbaceum |
| Cotton, Long Silk, also | Malvaceae | Gossypium barbadense |
| Long Staple, Sea Island | | |
| Cotton, Mexican, also | Malvaceae | Gossypium hirsutum |
| Short Staple | | |
| Soybean, Soya | Leguminosae | Glycine max |
| Sugar beet | Chenopodiaceae | Beta vulgaris altissima |
| Sugar cane | Woody-plant | Arenga pinnata |
| Tomato | Solanaceae | Lycopersicon esculentum |
| Tomato, Cherry | Solanaceae | Lycopersicon esculentum |
| | | cerasiforme |
| Tomato, Common | Solanaceae | Lycopersicon esculentum |
| | | commune |
| Tomato, Currant | Solanaceae | Lycopersicon pimpinellifolium |
| Tomato, Husk | Solanaceae | Physalis ixocarpa |
| Tomato, Hyenas | Solanaceae | Solanum incanum |
| Tomato, Pear | Solanaceae | Lycopersicon esculentum |
| | | pyriforme |
| Tomato, Tree | Solanaceae | Cyphomandra betacea |
| Potato | Solanaceae | Solanum tuberosum |
| Potato, Spanish, Sweet | Convolvulaceae | Ipomoea batatas |
| potato | | |
| Rye, Common | Pooideae | Secale cereale |
| Rye, Mountain | Pooideae | Secale montanum |
| Pepper, Bell | Solanaceae | Capsicum annuum grossum |
| Pepper, Bird, also | Solanaceae | Capsicum annuum minimum |
| Cayenne, Guinea | | |
| Pepper, Bonnet | Solanaceae | Capsicum sinense |
| Pepper, Bulinose, also | Solanaceae | Capsicum annuum grossum |
| Sweet | | |
| Pepper, Cherry | Solanaceae | Capsicum annuum cerasiforme |
| Pepper, Cluster, also Red | Solanaceae | Capsicum annuum fasciculatum |
| Cluster | | |
| Pepper, Cone | Solanaceae | Capsicum annuum conoides |
| Pepper, Goat, also Spur | Solanaceae | Capsicum frutescens |
| Pepper, Long | Solanaceae | Capsicum frutescens longum |
| Pepper, Oranamental | Solanaceae | Capsicum annuum abbreviatum |
| Red, also Wrinkled | | |
| Pepper, Tabasco Red | Solanaceae | Capsicum annuum conoides |
| Lettuce, Garden | Compositae | Lactuca sativa |
| Lettuce, Asparagus, also | Compositae | Lactuca sativa asparagina |
| Celery | | |
| Lettuce, Blue | Compositae | Lactuca perennis |
| Lettuce, Blue, also | Compositae | Lactuca pulchella |
| Chicory | | |
| Lettuce, Cabbage, also | Compositae | Lactuca sativa capitata |
| Head | | |
| Lettuce, Cos, also | Compositae | Lactuca sativa longifolia |
| Longleaf, Romaine | | |
| Lettuce, Crinkle, also | Compositae | Lactuca sativa crispa |
| Curled, Cutting, Leaf | | |
| Celery | Umbelliferae | Apium graveolens dulce |
| Celery, Blanching, also | Umbelliferae | Apium graveolens dulce |
| Garden | | |
| Celery, Root, also | Umbelliferae | Apium graveolens rapaceum |
| Turniprooted | | |
| Eggplant, Garden | Solanaceae | Solanum melongena |
| Sorghum | Sorghum | All crop species |
| Alfalfa | Leguminosae | Medicago sativum |
| Carrot | Umbelliferae | Daucus carota sativa |
| Bean, Climbing | Leguminosae | Phaseolus vulgaris vulgaris |
| Bean, Sprouts | Leguminosae | Phaseolus aureus |
| Bean, Brazilian Broad | Leguminosae | Canavalia ensiformis |
| Bean, Broad | Leguminosae | Vicia faba |
| Bean, Common, also | Leguminosae | Phaseolus vulgaris |
| French, White, Kidney | | |
| Bean, Egyptian | Leguminosae | Dolichos lablab |
| Bean, Long, also | Leguminosae | Vigna sesquipedalis |
| Yardlong | | |
| Bean, Winged | Leguminosae | Psophocarpus tetragonolobus |
| Oat, also Common, Side, | Avena | Sativa |
| Tree | | |
| Oat, Black, also Bristle, | Avena | Strigosa |
| Lopsided | | |
| Oat, Bristle | Avena | |
| Pea, also Garden, Green, | Leguminosae | Pisum, sativum sativum |
| Shelling | | |
| Pea, Blackeyed | Leguminosae | Vigna sinensis |
| Pea, Edible Podded | Leguminosae | Pisum sativum axiphium |
| Pea, Grey | Leguminosae | Pisum sativum speciosum |
| Pea, Winged | Leguminosae | Tetragonolobus purpureus |
| Pea, Wrinkled | Leguminosae | Pisum sativum medullare |
| Sunflower | Compositae | Helianthus annuus |
| Squash, Autumn, Winter | Dicotyledoneae | Cucurbita maxima |
| Squash, Bush, also | Dicotyledoneae | Cucurbita pepo melopepo |
| Summer | | |
| Squash, Turban | Dicotyledoneae | Cucurbita maxima |
| | | turbaniformis |
| Cucumber | Dicotyledoneae | Cucumis sativus |
| Cucumber, African, also | | Momordica charantia |
| Bitter | | |
| Cucumber, Squirting, | | Ecballium elaterium |
| also Wild | | |
| Cucumber, Wild | | Cucumis anguria |
| Poplar, California | Woody-Plant | Populus trichocarpa |
| Poplar, European Black | | Populus nigra |
| Poplar, Gray | | Populus canescens |
| Poplar, Lombardy | | Populus italica |
| Poplar, Silverleaf, also | | Populus alba |
| White | | |
| Poplar, Western Balsam | | Populus trichocarpa |
| Tobacco | Solanaceae | Nicotiana |
| Arabidopsis Thaliana | Cruciferae | Arabidopsis thaliana |
| Turfgrass | Lolium | |
| Turfgrass | Agrostis | |
| | Other families of turfgrass | |
| Clover | Leguminosae | |

The compounds identified by the method of the present invention may be useful for identifying complementation mutants of the conidiation and/or sporulation pathway. In one example, if a particular compound identified by the methods of the present invention specifically inhibits a particular enzyme in the conidiation pathway, for example, random or directed mutants could be generated, and incubated in the presence of a compound of the present invention, and the surviving mutants isolated and characterized. The mutants that survive would likely represent complementation mutants of the conidiation and/or sporulation pathway. The methods of generating mutants are known in the art, but may include incubation with a chemical mutagen, such as DMS or EMS, or UV irradiation, for example. Additional methods of generating mutants may be found in Sambrook J. L., et al., Molecular cloning. A Laboratory Manual. (2nd ed.) Cold Spring Harbor Laboratory Press, Cold Spring Harbor, NY, (1989); and in, “Fungal Genetics”, Bos, C.J.,ed, Chapter 2, Marcel Dekker, Inc., New York, pp. 13-42, (1996)).

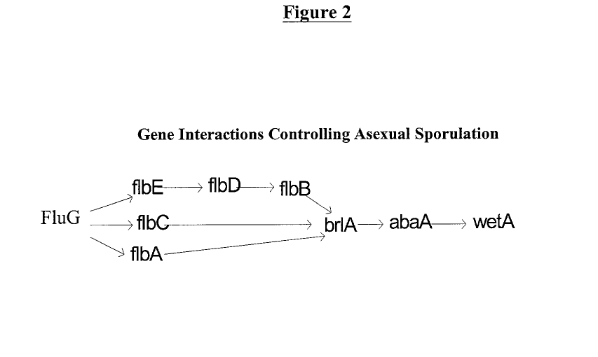

In another example, the compounds identified by the present invention are useful for selectively studying a particular conidiation sub-pathway. For example, if a compound identified by the methods of the present invention specifically inhibits a particular enzyme in one of the condiation sub-pathways (see FIG. 2), and the sample is incubated in the presence of the inhibitor, such an inhibitor may enable selective characterization of one or more of the other sub-pathways as the targeted sub-pathway would no longer be functional, for example. Specifically, a compound identified by the method of the present invention may inhibit the flbE gene. In such an instance, the flbC and/or flbA sub-pathways may be favored, due to the inhibition of the flbE-to-flbD-to-flbB sub-pathway, for example.

In yet another example, more than one compound identified by the method of the present invention may be combined to facilitate characterization of a specific conidiation sub-pathway, in addition to facilitating the possible isolation of complementation mutants of such a pathway. For example, one compound identified by the method of the present invention may inhibit the flbE, while another compound may inhibit the flbC. Thus, by combining the two compounds and treating a sample, the flbA sub-pathway may be favored as the flbC and the flbE-to-flbD-to-flb-B sub-pathways would be inhibited, for example.

The compounds identified by the method of the present invention are useful as a reagent in a diagnostic kit. For example, a compound of the present invention may inhibit a particular enzyme in a pathway for sporulation and/or conidiation. Since the particular enzyme may exist in several allelic forms, the compound may inhibit only one particular allelic form, thus enabling rapid identification of which form of the enzyme a particular fungus is expressing or responsible for a particular phenotype or trait, for example. Alternatively, specific mutations may exist in the general fungal populations that are either sensitive or resistant to the compound of the present invention. Therefore, rapid identification of the fungal species harboring the mutant or wild type form of the enzyme may be possible.

Thus, the present invention relates to a method of using an inhibitor of the present invention in a diagnostic kit comprising: a) providing a test kit comprising a solid support comprising growth medium and a fungal agent, b) culturing the fungal agent on solid support, c) adding a test compound to the medium from step “a”, and d) identifying compounds that suppress fungal conidiation by assessing whether the compound from step “c” inhibits the formation of conidia.

The sample may be a member selected from the group consisting of: fungal organism, fungal tissue, fungal cells, fungal protein, fungal DNA, fungal RNA, fungal cDNA, fungal genomic DNA, etc. Alternatively, the sample may be a member selected from the group consisting of: plants, plant tissue, plant cells, plant protein, plant DNA, plant RNA, plant cDNA, plant genomic DNA, etc of one of the fungi selected from the group consisting of Aspergillus, Penicillium, Botrytis, and Alternaria. Preferably, the fungal agent of the present invention is Aspergillus nidulans. Other fungal species may be substituted for the fungal agents of the present invention, particularly other species of, Aspergillus like Aspergillus niger, Aspergillus fumigatus, Aspergillusfiavus, Aspergillus oryzae. Additional suitable species are known in the art and are described elsewhere herein.e, preferably Aspergillus nidulans . Alternatively, the sample may comprise any combination of the above. The solid support may be for example a petri dish, a square bioassay plate or a glass microscope slide.

The sample may be any plant and/or fungus described herein and known in the art.

Formulations

Compounds identified by the method of the present invention may generally be used as a formulation or composition with an agriculturally suitable carrier comprising at least one of a liquid diluent, a solid diluent or a surfactant. The formulation or composition ingredients are selected to be consistent with the physical properties of the active ingredient, mode of application and environmental factors such as soil type, moisture and temperature. Preferably, the formulations comprise, as an active ingredient, one or more compounds identified by the method of the present invention. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations may typically contain effective amounts of active ingredient, diluent and surfactant within the approximate ranges that add up to 100 percent by weight as provided in the Table below.

| | Active | Diluent | Surfactant |
|---|---|---|---|
| Formulation | Ingredient (wt %) | (wt %) | (wt %) |
| Water-Dispersible and | 5-90 | 0-94 | 1-15 |
| Water-soluble, Granules, Tablets | | | |
| and Powders | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typicalliquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxyethylene/polyoxypropylene block copolymers. Solid diluents include, for example, clays such as bentonite, montinorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084.

Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

The compounds identified by the method of the present invention may be useful as plant disease control agents. The compounds identified by the method of the present invention therefore further comprises a method for controlling plant diseases caused by fungal plant pathogens comprising applying to the plant or portion thereof to be protected, or to the plant seed or seedling to be protected, an effective amount of a compound of the invention or a fungicidal composition containing said compound. The compounds and compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, vegetable, field, cereal, and fruit crops. These pathogens include Plasmopara viticola, Phytophthora infestans, Peronospora tabacina, Pseudoperonospora cubensis, Pythium aphanidermatum, Alternaria brassicae, Septoria nodorum, Septoria tritici, Cercosporidium personatum, Cercospora arachidicola, Pseudocercosporella herpotrichoides, Cercospora beticola, Botrytis cinerea, Moniliniafructicola, Pyricularia oryzae, Podosphaera leucotricha, Venturia inaequalis, Erysiphe graminis, Uncinula necatur, Puccinia recondita, Puccinia graminis, Hemileia vastatrix, Puccinia striformis, Puccinia arachidis, Rhizoctonia solani, Sphaerotheca fuliginea, Fusarium oxysporum, Verticillium dahliae, Pythium aphanidennatum, Phytophthora megasperma, Sclerotinia sclerotiorum, Sclerotium rolfsii, Erysiphe polygoni, Pyrenophora teres, Gaeumannomyces gramins, Rynchosporium secalis, Fusarium roseum, Bremia lactucae and other generea and species closely related to these pathogens.

Compounds identified by the method of the present invention can also be mixed with one or more other insecticides, fungicides, nematicides, bactericides, acaricides, growth regulators, chemosterilants, semiochemicals, repellents, attractants, pheromones, feeding stimulants or other biologically active compounds to form a multi-component pesticide giving an even broader spectrum of agricultural protection. Examples of such agricultural protectants with which compounds identified by the method of the present invention can be formulated are: insecticides such as abamectin, acephate, azinphos-methyl, bifenthrin, buprofezin, carbofuran, chlorpyrifos, chlorpyrifos-methyl, cyfluthrin, beta-cyfluthrin, deltamethrin, diafenthiuron, diazinon, diflubenzuron, dimethoate, esfenvalerate, fenpropathrin, fenvalerate, fipronil, flucythrrinate, tau-fluvalinate, fonophos, imidacloprid, isofenphos, malathion, metaldehyde, methamidophos, methidathion, methomyl, methoprene, methoxychlor, monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; fungicides such as azoxystrobin (ICIA5504), benomyl, blasticidin-S, Bordeaux mixture (tribasic copper sulfate), bromuconazole, captafol, captan, carbendazim, chloroneb, chlorothalonil, copper oxychloride, copper salts, cymoxanil, cyproconazole, cyprodinil (CGA 219417), diclomezine, dicloran, difenoconazole, dimethomorph, diniconazole, diniconazole-M, dodine, edifenphos, epoxyconazole (BAS 480F), fenarimol, fenbuconazole, fenpiclonil, fenpropidin, fenpropimorph, fluquinconazole, flusilazole, flutolanil, flutriafol, folpet, fosetyl-aluminum, furalaxyl, hexaconazole, ipconazole, iprobenfos, iprodione, isoprothiolane, kasugamycin, kresoxim-methyl (BAS 490F), mancozeb, maneb, mepronil, metalaxyl, metconazole, myclobutanil, neo-asozin (ferric methanearsonate), oxadixyl, penconazole, pencycuron, probenazole, prochloraz, propiconazole, pyrifenox, pyroquilon, sulfuir, tebuconazole, tetraconazole, thiabendazole, thiophanate-methyl, thiram, triadimefon, triadimenol, tricyclazole, triticonazole, uniconazole, validamycin and vinclozolin; nematocides such as aldoxycarb and fenamiphos; bactericides such as streptomycin; acaricides such as amitraz, chinomethionat, chlorobenzilate, cyhexatin, dicofol, dienochlor, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi.

Such formulations may also contain more than one active compound identified by the method of the present invention, though preferably at least one, two, three, four, or more. The purpose for combining more than one active compound identified by the method of the present invention may be to take advantage of each compounds unique characteristics. For example, one compound may have very high activity, though may be inactive in the presence of water (e.g., rain, etc.),while another compound, for example, may have less activity, though is more resistant to the presence of water. Thus, a combination of both compounds would ensure improved efficacy under a variety of conditions.

In certain instances, combinations with other fungicides having a similar spectrum of control but a different mode of action will be particularly advantageous for resistance management.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to the seed to protect the seed and seedling.

Rates of application for these compounds can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

The present compositions may be prepared in a known manner, for example by homogeneously mixing or grinding the active ingredient(s) with other ingredients. Additional components may be admixed with the composition at any point during the process, including during and/or after any mixing step of the herbicide components.

Experimental:

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, percent is percent by weight given the component and the total weight of the composition, temperature is in ° C. or is at ambient temperature, and pressure is at or near atmospheric.

### EXAMPLES

### Example 1

Primary Screen for Antisporulant Activity Using a Wildtype Strain of Aspergillus nidulans growing in vitro.

A. nidulans strain 003 (ATCC 36321) was cultured as follows. A fresh spore suspension was prepared by growing Aspergillus nidulans on potato dextrose agar (PDA) or yeast extract glucose agar (YAG) in Petri dishes for 3 days at 37° C. The conidia were harvested by rinsing the plates with 0.1% Tween 80 and a small quantity of glass beads. The conidial suspension was centrifuged and stored in phospate buffered saline (PBS) solution at a concentration of ca. 1×10⁷ spores per ml as determined with a hemocytometer slide. The suspension of conidia stored at 4° C. in PBS solution was stable for more than 1 year.

Reagents

Phosphate buffered saline solution contained 0.9% (w:v) NaCl in 10 mM sodium phosphate at pH7.4. Potato dextrose agar (PDA) was prepared by combining:

| | | |
|---|---|---|
| Difco potato dextrose agar (dehydrated) | 39 | g |
| Distilled water | 1000 | mL |

and autoclaving for 15 minutes at 120° C.

Yeast extract glucose agar (YAG) was prepared by combining:

| | | |
|---|---|---|
| Difco yeast extract | 5 | g |
| Glucose | 20 | g |
| Difco agar | 20 | g |
| Distilled water | 1000 | mL |

and autoclaving for 15 minutes at 120° C.

The growth medium used in the screen to identify compounds exhibiting antisporulant activity was PDA. The fungal innoculum was a spore suspension isolated from A. nidulans strain 003 with a spore concentration of 1×10⁷ spores per mL. In preparation for the screen, one part spore suspension was added to 1000 parts molten PDA (45-50° C.).

For the screen, Petri plates or square bioassay plates were poured and the medium containing the spore innoculum allowed to solidify prior to the application of test samples. The test samples were applied either on filter paper discs (7 mm diam.), or directly to the surface of the agar medium. Alternatively, the test samples were mixed directly in the agar medium. Each screen included a positive control treatment designed to provide a clear response (e.g., 5 μg of one of the active benzophenones described elsewhere herein). Plates were incubated for 2 days at 37° C. Positives were initially identified by the presence of a discolored zone around the test sample, and were confirmed by observation under a low power microscope (×20 magnification).

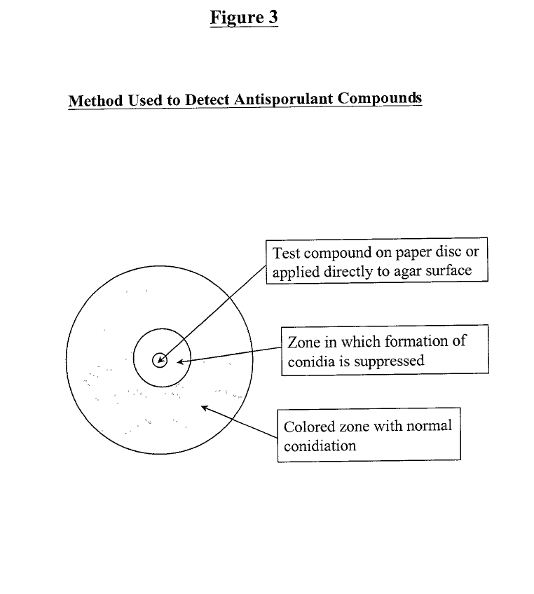

Mycelial growth was normal but spore production was greatly reduced (though not totally inhibited) compared to the normal growth area of the plate (FIG. 3). Cleistothecia, the sexual fruiting bodies, become visible on the agar surface at about 7 days after the start of the assay. The formation of cleistothecia was stimulated by positive test samples, and many more were present in the discolored zone around the test sample compared with the zone of normal growth.

Two panels of compounds, one composed of 22 known commercial fungicides with diverse mechanisms of action (Table 1), and a second composed of 30 benzophenones (Table 2) with the generalized structure (I), were tested using the screen. The compounds were tested by paper disc diffusion assay at a rate of 20μg per disc.

Table 1. Panel Of Known Commercial Fungicides With Diverse Sites Of Action. This table presents a list of 22 commercial agrochemical fungicides and antimycotics that were tested in the sporulation assay of the invention using Aspergillus nidulans. The corresponding sites of action within the fungal cell are also given. ‘Active’ compounds gave a discolored zone □ 15 mm in diameter including the diameter of the filter paper disc. ‘Moderately active’ compounds gave a discolored zone 10-14 mm in diameter including the diameter of the filter paper disc. For 'inactive'compounds, the discolored zone was <10 mm including the diameter of the filter paper disc or was completely absent. As shown, none of the commercial herbicides or antimycotics tested positive in the sporulation assay of the present invention—emphasizing the novel mode of action of the compounds for which the sporulation assay was designed to identify, which includes, for example, compound “A”. Further information on the biochemical mode of action of antifungal compounds is provided in Target Sites of Fungicide Action (ed. W. Köller, CRC Press, 1992).

TABLE 1
Panel Of Known Commercial Fungicides
With Diverse Sites Of Action.

| | | | Moderately | |
|---|---|---|---|---|
| Fungicide | Site of action | Active | active | Inactive |
| U-18666 | ergosterol | No | No | Yes |
| | biosynthesis | | | |
| | (squalene | | | |
| | cyclase) | | | |
| Tolnaftate | ergosterol | No | No | Yes |
| | biosynthesis | | | |
| | (squalene | | | |
| | monooxygenase) | | | |
| Ketoconazole | ergosterol | No | No | Yes |
| | biosynthesis | | | |
| | (lanosterol 14α- | | | |
| | demethylase) | | | |
| Metconazole | ergosterol | No | No | Yes |
| | biosynthesis | | | |
| | (lanosterol 14α- | | | |
| | demethylase) | | | |
| Fenarimol | ergosterol | No | No | Yes |
| | biosynthesis | | | |
| | (lanosterol 14α- | | | |
| | demethylase) | | | |
| Imazalil | ergosterol | No | No | Yes |
| | biosynthesis | | | |
| | (lanosterol 14α- | | | |
| | demethylase) | | | |
| Fenpropidin | ergosterol | No | No | Yes |
| | biosynthesis | | | |
| | (sterol Δ-14 | | | |
| | reductase) | | | |
| Tridemorph | ergosterol | No | No | Yes |
| | biosynthesis | | | |
| | (sterol Δ-14 | | | |
| | reductase) | | | |
| Amphotericin B | plasma | No | No | Yes |
| | membrane | | | |
| | (polyene) | | | |
| Cycloheximide | protein | No | No | Yes |
| | biosynthesis | | | |
| Polyoxin D | chitin | No | No | Yes |
| | biosynthesis | | | |
| | (cell wall) | | | |
| Nikkomycin | chitin | No | No | Yes |
| | biosynthesis | | | |
| | (cell wall) | | | |
| Metalaxyl | rRNA | No | No | Yes |
| | biosynthesis | | | |
| Vinclozolin | lipid | No | No | Yes |
| | peroxidation, | | | |
| | NADPH- | | | |
| | cytochrome c | | | |
| | reductase | | | |
| Benomyl | microtubule | No | No | Yes |
| | polymerization | | | |
| Diethofencarb | microtubule | No | No | Yes |
| | polymerization | | | |
| Carboxin | succinate | No | No | Yes |
| | dehydrogenase | | | |
| 5-F-Cytosine | nucleotide | No | No | Yes |
| | metabolism | | | |
| Azoxystrobin | respiration | No | No | Yes |
| Pyrimethanil | secretion of cell | No | No | Yes |
| | wall degrading | | | |
| | enzymes | | | |
| Chlorothalonil | multisite | No | No | Yes |
| Quinoxifen | Inhibition of G- | No | No | Yes |
| | protein | | | |
| | Regulation | | | |

With the exceptions of metalaxyl and amphotericin B, all of the compounds in Table 1 inhibit mycelial growth and, as a consequence, sporulation. None of the compounds had a stimulatory effect on the development of cleistothecia. More importantly, none of the commercial herbicides or antimycotics tested using the method of the present invention inhibited sporulation in the absence of inhibiting vegetative growth—emphasizing the novel mode of action of the compounds for which the sporulation assay was designed to identify, which includes, for example, compound “A”. Low power microscope observations confirmed the above observations for each tested compound. Criteria for activity (i.e., Active, Moderately Active, and Inactive) are as follows:

‘Active’ compounds gave a discolored zone ≧15 mm in diameter including the diameter of the filter paper disc. ‘Moderately active’ compounds gave a discolored zone 10-14 mm in diameter including the diameter of the filter paper disc. For ‘inactive’ compounds, the discolored zone was <10 mm including the diameter of the filter paper disc or was completely absent.

Of the benzophenones tested, 4 were active, 13 were moderately active, and 13 were inactive (Table 2).

Table 2. Effect Of Benzophenones On Sporulation Of A. nidulans. This table presents a list of 30 benzophenones of general structure (I) that were tested in the sporulation assay using Aspergillus nidulans. 'Active'compounds gave a discolored zone ≧15 mm in diameter including the diameter of the filter paper disc. ‘Moderately active’ compounds gave a discolored zone 10-14 mm in diameter including the diameter of the filter paper disc. For ‘inactive’ compounds, the discolored zone was<10 mm including the diameter of the filter paper disc or was completely absent.

TABLE 2
Effect Of Benzophenones On Sporulation Of A. nidulans.

| Active | Moderately Active | Inactive |
|---|---|---|
| Compounds | Compounds | Compounds |
| L | O | B |
| G | E | V |
| M | H | D |
| N | I | W |
| | J | F |
| | P | X |
| | Q | Y |
| | R | Z |
| | S | AA |
| | T | BB |
| | K | CC |
| | FF | DD |
| | U | EE |

For high-throughput screens, 245mm square bioassay dishes were poured and the medium containing the spore inoculum was allowed to solidify prior to application of test samples. Test samples, representing a broad spectrum of diverse chemical structures, were applied to the surface of the agar medium either by hand, using a replicator, or by robotic application. Each screen included a positive control treatment (e.g., 5 ∝g of one of the active benzophenones described elsewhere herein) that provided a positive response. Plates were incubated for 2 days at 37° C. Positives were initially identified by the presence of a discolored zone around the test sample. The positives were confirmed by retest followed by observation under a low power microscope (×20 magnification). Mycelial growth was normal but spore production was greatly reduced (though not totally inhibited) compared to the normal growth area of the plate.

7000 compounds with diverse structures have been tested using the such a highthroughput screen. The compounds were tested by direct application to the medium using a replicator. The compound provided as compound A, shown below, tested positive for anti-sporulation activity.

### Example 2

Primary screen for antisporulant activity using wild-type strains of suitable filamentous fungi.

Filamentous fungi that sporulate freely in vitro, and can be readily maintained in the laboratory, may be used as test organisms as alternatives to A. nidulans Examples of suitable fungi include species of Penicillium, Botrytis, and Alternaria. Incubation temperatures for routine maintenance of cultures and during the conduct of tests may be varied to optimize growth depending on the fungus used.

Wild-type strains of Penicillium digitatum, Botrytis cinerea and Alternaria solani were cultured using routine methods known in the art. Briefly, fresh spore suspensions were prepared by growing the fungi on potato dextrose agar (PDA) in Petri dishes at room temperature until the cultures were covered with conidia. The conidia were harvested by rinsing the plates with 0. 1% Tween 80 in water and a small quantity of glass beads. The conidial suspensions were centrifuged and stored in phosphate buffered saline (PBS) solution at a concentration of ca. 1×10⁷ spores per ml as determined with a hemocytometer slide. The suspension of conidia stored at 4° C. in PBS solution was stable for more than 1 year.

The preparation of phosphate buffered saline (PBS) and potato dextrose agar (PDA) is described elsewhere herein.

The medium used in the screen to identify compounds exhibiting antisporulant activity was PDA. The fungal inoculum was a spore suspension isolated from wild-type strains of Penicillium digitatum, or Botrytis cinerea, or Alternaria solani, with a spore concentration of 1×10⁷ spores per mL. In preparation for the screen, one part spore suspension was added to 1000 parts molten PDA (45-50° C.).

For the screen, Petri plates or 245 mm square bioassay dishes were poured and the medium containing the spore inoculum was allowed to solidify prior to the application of test samples. The test samples were applied either on filter paper discs (7 mm diam.), or directly to the surface of the agar medium. Each screen included a positive control treatment (e.g., 20 ∝g of one of the active benzophenones described elsewhere herein) that provided a positive response. Plates were incubated for 5 days at room temperature. Positives were initially identified by the presence of a discolored zone around the test sample, and were confirmed by observations under a low power microscope (×20 magnification). Mycelial growth was normal, but spore production was greatly reduced (though not totally inhibited) when compared to the normal growth area of the plate.

### Example 3

The use of a Sporulation Mutant of A. nidulans to Better Define the Point in the Developmental Pathway at Which an Active Compound Exerts its Effect.

Mutant strains of A. nidulans are commercially available which are blocked at known points in the pathway that regulates conidiation (FIG. 2). By studying the effects of a test compound on asexual reproduction in these strains, it is possible to delineate at which point in the pathway the compound acts.

The gene br/A (‘bristle’phenotype) has a central role in conidiation of A. nidulans (Miller B. L. (1993). Trends Genet. 9, 293-295). A null mutant brlA 23 was

The gene brlA (‘bristle’ phenotype) has a central role in conidiation of A. nidulans (Miller B. L. (1993). Trends Genet. 9, 293-295). A null mutant briA 23 was obtained from Dr. J. Clutterbuck, University of Glasgow, Scotland. The effect of a representative active benzophenone on brlA 23 was determined by a disc diffusion assay on PDA growth medium. A suspension of hyphal fragments in water was prepared from a culture grown on unamended medium, mixed with molten PDA, and poured into a Petri dish. Once the medium solidified, the disc impregnated with the active benzophenone was placed on the agar and the plate incubated at 37° C. for one week. After the incubation period, the formation of a zone of differential growth around the treated disc is indicative that the mutant strain ofA. nidulans is sensitive to the same test compound previously identified as active on the wild-type strain. Therefore, this observation suggests the site of action of the test compound is upstream of the brl gene in the pathway illustrated in FIG. 2.

### Example 4

The use of Bioassays on Microscope Slides to Facilitate Examination of Morphological Effects of Test Compounds on Fungal Morphology including conidiation.

Characterization of morphological effects of test compounds that suppress sporulation can be made initially by observation of fungal colonies under a low power microscope. This may be confirmed by detailed examination by light microscopy at higher power or by scanning electron microscopy, after excision of the fungus from regions of the colony exhibiting differential growth e.g., in a disc diffusion assay from an area immediately around the disc, compared to an area remote from the disc. Alternatively, detailed microscopic characterization may be greatly facilitated by undertaking assays directly on microscope slides and making observations on specimens without any physical interference with the medium or organism.

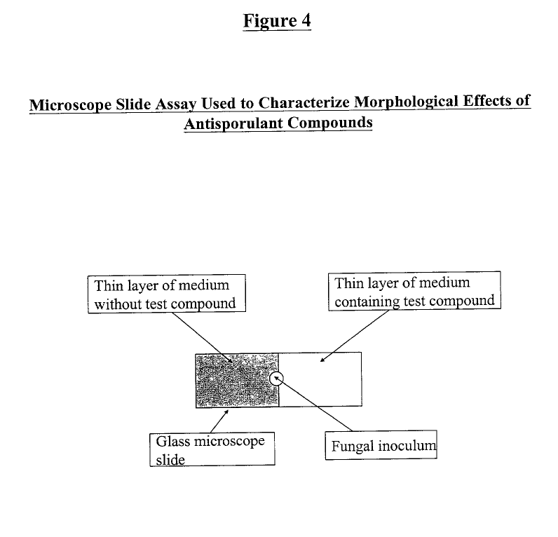

One half of a microscope slide was coated with a thin layer (ca lmm) of unamended PDA and the agar allowed to solidify. The other half of the slide was coated with a layer of comparable thickness of molten PDA amended with a representative active benzophenone. After the medium gelled on both halves of the fragments, or a mycelial disc of the fungus. Slides were incubated in a moist chamber at 37° C., and observations made by light microscopy after 2-3 days (see FIG. 4).

The use of bioassays on microscope slides to facilitate

In a wild-type strain of A. nidulans there was abundant conidiation on the side of the slide without the test compound. For reference standard, normal conidiophores appear and consist equentially of a stalk, a swollen distal vesicle, and specialized cells termed sterigmata upon which the spores or conidia are borne. By contrast, there were many fewer conidia on the conidiophores produced on the treated side of the slide. Furthermore, the conidiophores produced on treated medium were often aberrant in having multi-tiered layers of sterigmata.

### Example 5

Control of Agronomically Important Diseases of Plants by Compounds Identified as Inhibitors of Asexual Sporulation by the Method of the Present Invention.

Test compounds were evaluated for efficacy in controlling a diverse range of important fungal diseases of plants including wheat powdery mildew, apple scab, rice blast, and wheat Leptosphaeria (Septoria) nodorum blotch (Table 3).

Table 3. Control of Plant Diseases by Benzophenones of General Structure (I). This table presents data on levels of control of plant diseases (wheat powdery mildew, wheat Leptosphaeria nodorum, apple scab, and rice blast) from treatment with 30 benzophenones of general structure (I). Plants were treated with the compounds by spray application to the leaves, inoculated with the appropriate fungal pathogens, and incubated under environmental conditions favoring development of disease. Levels of disease in treated plants were compared to those in untreated but incoculated controls. Disease control is indicated as follows: “+++” =high control; “++” =moderate control; “+” =low control; and “0”=no control. ‘n.t.’ indicates ‘not tested’.

TABLE 3
Control of Plant Diseases by Benzophenones of General Structure (I).

| | | Level of Plant | | | |
|---|---|---|---|---|---|
| | | Disease Control | | | |
| | | Wheat Powdery | Wheat | Apple | Rice |
| Compound | Structure | Mildew | L. nodorum | Scab | Blast |
| B | 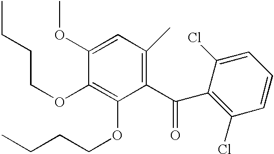 | +++ | 0 | 0 | 0 |
| C | 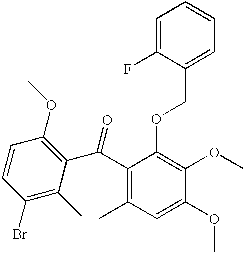 | +++ | n.t. | 0 | ++ |
| D | 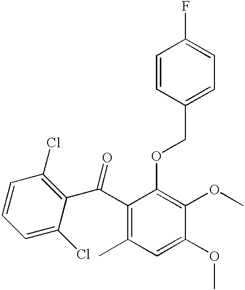 | +++ | n.t. | n.t. | n.t. |
| E | 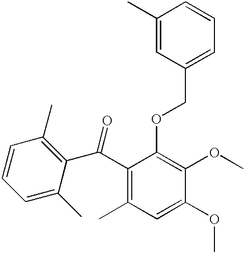 | +++ | n.t. | n.t. | n.t. |
| F | 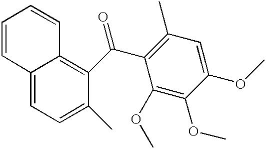 | +++ | 0 | 0 | ++ |
| G | 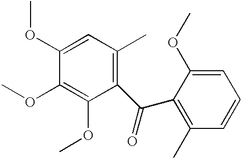 | +++ | 0 | 0 | 0 |
| H | 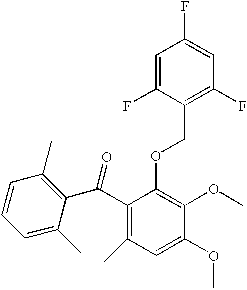 | +++ | 0 | 0 | 0 |
| I | 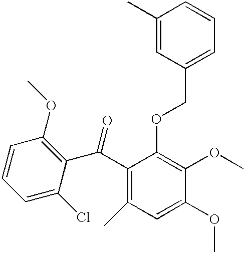 | +++ | 0 | ++ | + |
| J | 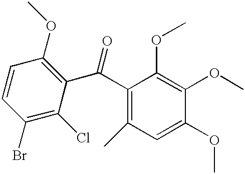 | +++ | 0 | + | 0 |
| K | 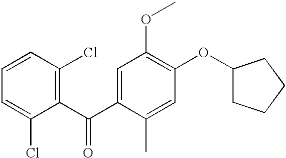 | 0 | +++ | n.t. | 0 |
| L | 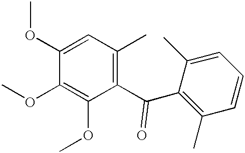 | +++ | 0 | 0 | 0 |
| M | 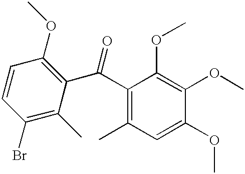 | +++ | 0 | +++ | 0 |
| N | 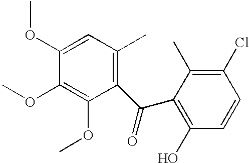 | +++ | 0 | 0 | ++ |
| O | 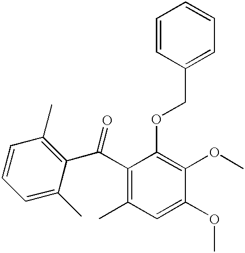 | +++ | 0 | 0 | ++ |
| P | 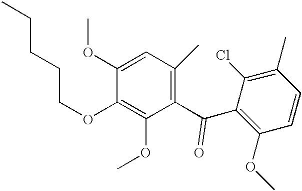 | +++ | 0 | 0 | ++ |
| Q | 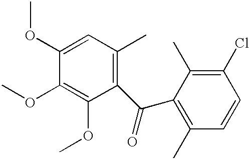 | +++ | 0 | + | ++ |
| R | 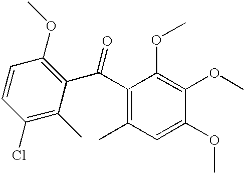 | +++ | 0 | 0 | ++ |
| S | 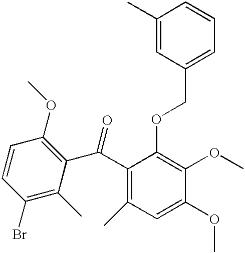 | +++ | n.t. | n.t. | n.t. |
| T | 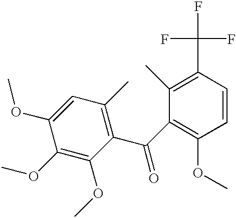 | +++ | 0 | 0 | 0 |
| U | 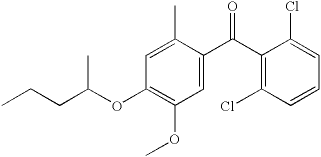 | ++ | +++ | 0 | ++ |
| V | 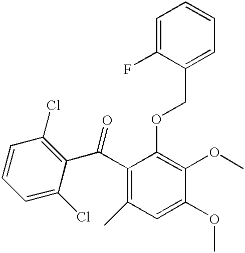 | +++ | 0 | 0 | ++ |
| W | 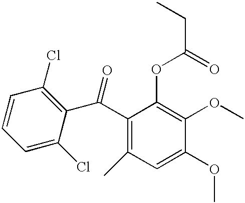 | +++ | 0 | +++ | +++ |
| X | 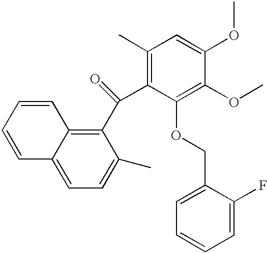 | +++ | 0 | 0 | 0 |
| Y | 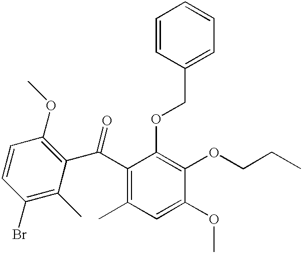 | +++ | 0 | 0 | 0 |
| Z | 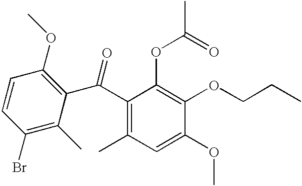 | 0 | ++ | 0 | 0 |
| AA | 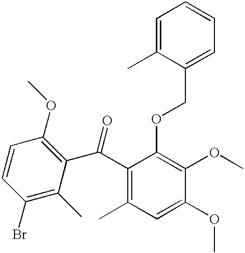 | +++ | 0 | 0 | 0 |
| BB | 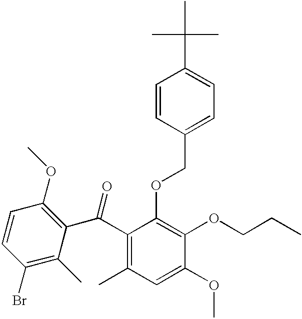 | +++ | 0 | 0 | 0 |
| CC | 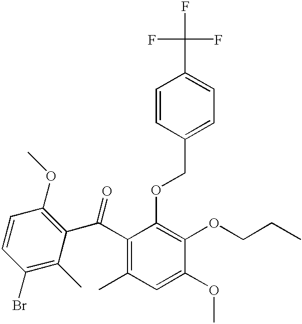 | +++ | 0 | 0 | 0 |
| DD | 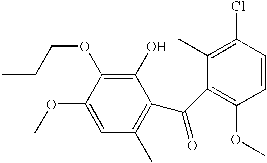 | +++ | 0 | 0 | 0 |
| EE | 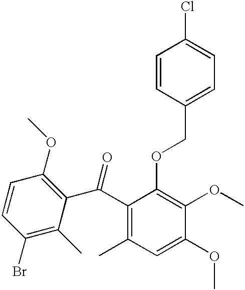 | +++ | 0 | 0 | 0 |

Activity Against Wheat Powdery Mildew (Blumeria [Erysiphe] graminis f.sp. tritici)

This test measures the protectant activity of test compounds applied as a foliar spray. Wheat seeds (cv. Kanzler) are sown in 2.25-inch square plastic pots containining Metro-Mix 350 (W. R. Grace Co.) and grown in a greenhouse to the primary leaf stage (ca. 1 week old). Test compounds are applied to leaves to runoff by spraying in a solvent/surfactant system containing 5% acetone and 0.05% Tween 20 in deionized water. Plants are allowed to air-dry for 2-5 hours, and then inoculated by dusting with conidia of B. graminis f.sp. tritici. Inoculated plants are maintained in a greenhouse until disease symptoms are observed on untreated plants. Disease evaluations are typically made at 7 days after inoculation.

b) Activity Against Apple Scab (Venturia inaequalis)

This test measures the protectant activity of test compounds applied as a foliar spray. Apple seeds (Captan 5WP-treated) are germinated in a flat containing perlite and vermiculite (1: 1, v/v). After vernalization to break dormancy, flats are maintained in a greenhouse for 1-2 weeks. Seedlings are then transplanted (3 per pot) into 3-inch square plastic pots containing Metro-Mix 350 and kept under greenhouse conditions. Approximately 2 weeks after transplanting, test compounds are applied to leaves to runoff by spraying in a solvent/surfactant system containing 5% acetone and 0.05% Tween 20 in deionized water. Plants are allowed to air-dry for 2-5 hours before inoculation of the leaves with a conidial suspension of the apple scab fungus, V. inaequalis. After inoculation, plants are initially maintained in a moisture chamber (22° C.; 100% relative humidity) for 2 days, before transfer to a greenhouse. Disease evaluations are made at 2 weeks after inoculation.

c) Activity Against Rice Blast (Magnaporthe grisea) This test measures the protectant activity of test compounds applied as a foliar spray. Rice seeds (approximately 20 per pot) are planted in 6 cm diameter plastic cups (two drainage holes per pot) containing pasteurized potting medium and maintained in a greenhouse. Approximately 2 weeks after transplanting, test compounds are applied to leaves to runoff by spraying in a solvent/surfactant system containing 5% acetone and 0.05% Tween 20 in deionized water. Plants are allowed to air-dry for 2-5 hours before inoculation of the leaves with a conidial suspension of the rice blast fungus, M grisea. After inoculation, plants are initially maintained in a moisture chamber (22° C.; 100% relative humidity) for 40 hours, before transfer to a greenhouse. Disease evaluations are made at 6-9 days after inoculation.

d) Activity Against Wheat L. nodorum Blotch

This test measures the protectant activity of test compounds applied as a foliar spray. Wheat seeds (approximately 20 per pot) are sown in 2.25-inch square plastic pots containining Metro-Mix 350 (W. R. Grace Co.) and maintained in a greenhouse to the primary leaf stage (ca. 1 week old). Test compounds are applied to leaves to runoff by spraying in a solvent/surfactant system containing 5% acetone and 0.05% Tween 20 in deionized water. Plants are allowed to air-dry for 2-5 hours before inoculation of the leaves with a conidial suspension of the leaf blotch fungus, L. nodorum. After inoculation, plants are initially maintained in a moisture chamber (22° C.; 100% relative humidity) for 48 hours, before transfer to a greenhouse. Disease evaluations are made at 7-9 days after inoculation.

The skilled artisan would appreciate that the sporulation inhibitor compounds identified by the method of the present invention may be useful as fungicides for a number of other fungal pathogens and diseases. Such pathogens and diseases are known in the art, some of which are described elsewhere herein. Moreover, the methods for testing the efficacy of a compound identified by the method of the present invention in controlling such fungal pathogens and/or diseases are known in the art. However, the following general method may be applied:

Plants are grown in a medium and are maintained under greenhouse conditions for a period of time. The plants are preferably one or more of the plants provided elsewhere herein and include mature plants, seedlings, plant tissue, plant seeds, and/or plant cells of these plants. The period of incubation will vary based upon the specific plant species and the maturity of the plant (e.g., plant seeds and tissues may require longer periods of incubation, as compared to plant seedlings, etc.). Test compounds are applied to the plant sample at a defined rate of a.i. by spraying in a solvent/surfactant system containing 5% acetone and 0.05% Tween 20 in deionized water. Plant samples may be allowed to air-dry for 2-5 hours before inoculation of the leaves with a conidial suspension of the test fungal pathogen. After inoculation, the plant sample is initially maintained in a moisture chamber (22° C.; 100% relative humidity) for 48 hours, before transfer to a greenhouse. Disease evaluations are made at 7-9 days after inoculation. As above, the specific conditions of this general method may vary according to the plant and/or fungal species and thus, is within the relative skill of the artisan.

Throughout this application, various publications are referenced, including, but not limited to, Adams, T. H., Wieser, J. K., & Yu,J.-H. (1998). Microbio. Mol. Biol. Rev. 62, 35-54; Adams, T. H. & Yu, J.-H. (1998). Curr. Opin. Microbioll, 674-677; Miller B. L. (1993). Trends Genet. 9, 293-295; Timberlake, W. E. & Marshall, M. A. (1988). Trends Genet. 4, 162-169. The disclosures of these publications, and all publications and other references cited herein, are hereby incorporated by reference in their entireties into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for identifying compounds that suppress fungal conidiation, wherein the method comprises:
a) culturing a fungal agent on a solid support comprising growth medium,
b) adding a test compound to the medium from step “a”, and
c) identifying compounds that suppress fungal conidiation by assessing whether the compound from step “b” inhibits the formation of conidia.

2. The method according to claim 1, wherein the compounds are assessed in step “c” by observing the formation of a discolored zone on the medium in an area proximate to the test compound.

3. The method according to claim 2, wherein the method further comprises d) determining whether spore production is reduced within the zone.

4. The method according to claim 3, wherein step “d” is performed by viewing the discolored zone under a miscroscope.

5. The method according to claim 3, wherein the method further comprises determining which test compounds have fungicidal effects.

6. The method according to claim 1, wherein the test compound inhibits one or more of the following genes: fluG, flbA, flbE, flbD, flbB, flbC, brlA, abaA, and wetA.

7. The method according to claim 1, wherein the fungal agent is Aspergillus, Penicillium, Botrytis, or Alternaria.

8. The method according to claim 1, wherein the solid support is a petri dish, a square bioassay plate, or a glass microscope slide.

9. A method of determining the mode of action of a compound that suppresses fungal conidiation, wherein the method comprises:
a) culturing a fungal agent on a solid support comprising growth medium,
b) adding a test compound to the medium from step “a”,
c) identifying compounds that suppress fungal conidiation by assessing whether the compound from step “b” inhibits the formation of conidia,
d) culturing a mutant fungal agent on a solid support,
e) adding the compound identified in step “c” to the cultured mutant fungal agent from step “d”, and
f) determining which mutant fungal agents are sensitive or resistant to the compound from step “c”.

10. The method according to claim 9, wherein the test compound inhibits one or more of the following genes: fluG, flbA, flbE, flbD, flbB, flbC, brlA, abaA, and weta.

11. The method according to claim 9, wherein the fungal agent in step “a” is Aspergillus, Penicillium, Botrytis, or Alternaria.

12. The method according to claim 9, wherein the solid support in step “a” is a petri dish, a square bioassay plate, or a glass microscope slide.

13. The method according to claim 9, wherein the mutant fungal agent in step “d”is fluG, flbA, flbE, flbD, flbB, flbC, brlA, abaA, wetA, stuA, or medA.

14. The method according to claim 9, wherein the fungal compound inhibits sporulation.

15. A method of using a diagnostic kit for analyzing compounds for suppression of fungal conidiation wherein the method comprises:
a) providing a test kit comprising a solid support comprising growth medium, and a fungal agent,
b) culturing the fungal agent on the solid support,
c) adding a test compound to the medium of from step “a”, and
d) identifying compounds that supress fungal conidiation by assessing whether the compound from step “c” inhibits the formation of conidia.

16. The kit according to claim 15, wherein the fungal agent comprises a fungal organism, fungal tissue, fungal cells, fungal protein, fungal DNA, fungal RNA, fungal cDNA, or fungal genomic DNA.

17. The kit according to claim 15, wherein the growth medium comprises a plant, plant tissue, plant cells, plant protein, plant DNA, plant RNA, plant cDNA, or plant genomic DNA.

18. The method according to claim 15, wherein the solid support is a petri dish, a square bioassay plate or a glass microscope slide.
